(19) United States
(12) Patent Application Publication
(10) Pub. No.: US 2018/0209874 A1
(43) Pub. Date: Jul. 26, 2018

(54) ASSAY PERFORMANCE SYSTEMS INCLUDING AQUEOUS SAMPLE STABILIZATION

(71) Applicant: Bio-Rad Laboratories, Inc., Hercules, CA (US)

(72) Inventors: Sean Cater, Richmond, CA (US); John Dzenitis, Danville, CA (US); Stefano Schiaffino, Pleasanton, CA (US); Pallavi Shah, Milpitas, CA (US); Andy Utada, Wayne, PA (US)

(21) Appl. No.: 15/881,559

(22) Filed: Jan. 26, 2018

Related U.S. Application Data
(60) Provisional application No. 62/451,004, filed on Jan. 26, 2017.

(51) Int. Cl. G01N 1/18; C12Q 1/6806; B01L 7/00

(52) U.S. Cl. CPC G01N 1/18; B01L 7/52; C12Q 1/6806

(57) ABSTRACT

An assay performance system may include modules configured to store aqueous sample plates, conduct droplet generation or emulsification of aqueous samples, and to perform thermocycling and droplet reading functions. One or more samples may be emulsified and stored in an emulsified state for extended times prior to thermocycling. Accordingly, the assay performance system may include material handling systems and methods to accommodate the storage function.

### CROSS-REFERENCES

This application claims the benefit under 35 U.S.C. § 119(e) of the priority of U.S. Provisional Patent Application Ser. No. 62/451,004, filed Jan. 26, 2017, the entirety of which is hereby incorporated by reference for all purposes.

The following related applications and materials are incorporated herein, in their entireties, for all purposes: U.S. Pat. No. 7,041,481; U.S. Pat. No. 9,089,844; U.S. Pat. No. 9,156,010; U.S. Patent Application Publication No. 2010/0173394 A1, published Jul. 8, 2010; U.S. Patent Application Publication No. 2012/0190032 A1, published Jul. 26, 2012; U.S. Patent Application Publication No. 2012/0194805 A1, published Aug. 2, 2012; U.S. Patent Application Publication No. 2012/0152369 A1, published Jun. 21, 2012; and Joseph R. Lakowicz, PRINCIPLES OF FLUORESCENCE SPECTROSCOPY (2nd Ed. 1999).

### INTRODUCTION

Many biomedical applications rely on high-throughput assays of samples combined with reagents. For example, in research and clinical applications, high-throughput genetic tests using target-specific reagents can provide high-quality information about samples for drug discovery, biomarker discovery, and clinical diagnostics, among others. As another example, infectious disease detection often requires screening a sample for multiple genetic targets to generate high-confidence results.

The trend is toward reduced volumes and detection of more targets. However, creating and mixing smaller volumes can require more complex instrumentation, which increases cost. Accordingly, improved technology is needed to permit testing greater numbers of samples and combinations of samples and reagents, at a higher speed, a lower cost, and/or with reduced instrument complexity.

Emulsions hold substantial promise for revolutionizing high-throughput assays. Emulsification techniques can create billions of aqueous droplets that function as independent reaction chambers for biochemical reactions. For example, an aqueous sample (e.g., 200 microliters) can be partitioned into droplets (e.g., four million droplets of 50 picoliters each) to allow individual sub-components (e.g., cells, nucleic acids, proteins) to be manipulated, processed, and studied discretely in a massively high-throughput manner.

Splitting a sample into droplets offers numerous advantages. Small reaction volumes (picoliters to nanoliters) can be utilized, allowing earlier detection by increasing reaction rates and forming more concentrated products. Also, a much greater number of independent measurements (thousands to millions) can be made on the sample, when compared to conventional bulk volume reactions performed on a micoliter scale. Thus, the sample can be analyzed more accurately (i.e., more repetitions of the same test) and in greater depth (i.e., a greater number of different tests). In addition, small reaction volumes use less reagent, thereby lowering the cost per test of consumables. Furthermore, microfluidic technology can provide control over processes used for the generation, mixing, incubation, splitting, sorting, and detection of droplets, to attain repeatable droplet-based measurements.

Aqueous droplets can be suspended in oil to create a water-in-oil emulsion (W/O). The emulsion can be stabilized with a surfactant to reduce or prevent coalescence of droplets during heating, cooling, and transport, thereby enabling thermal cycling to be performed. Accordingly, emulsions have been used to perform single-copy amplification of nucleic acid target molecules in droplets using the polymerase chain reaction (PCR).

Compartmentalization of single molecules of a nucleic acid target in droplets of an emulsion alleviates problems encountered in amplification of larger sample volumes. In particular, droplets can promote more efficient and uniform amplification of targets from samples containing complex heterogeneous nucleic acid populations, because sample complexity in each droplet is reduced. The impact of factors that lead to biasing in bulk amplification, such as amplification efficiency, G+C content, and amplicon annealing, can be minimized by droplet compartmentalization. Unbiased amplification can be critical in detection of rare species, such as pathogens or cancer cells, the presence of which could be masked by a high concentration of background species in complex clinical samples.

Despite their allure, emulsion-based assays present technical challenges for high-throughput testing, which can require creation of tens, hundreds, thousands, or even millions of individual samples and sample/reagent combinations. Thus, there is a need for improved techniques for the generation, mixing, incubation, splitting, sorting, and detection of droplets. Storage of aqueous samples for long periods of time while awaiting processing may lead to less-than-ideal reaction stabilities. Accordingly, there is a need for methods and systems that include improved stabilization of samples.

### SUMMARY

The present disclosure provides systems, apparatuses, and methods relating to assay performance systems including aqueous sample stabilization.

Features, functions, and advantages may be achieved independently in various embodiments of the present disclosure, or may be combined in yet other embodiments, further details of which can be seen with reference to the following description and drawings.

### BRIEF DESCRIPTION OF THE DRAWINGS

FIG. 1 is a flow chart listing illustrative steps that may be performed in a method of sample analysis using droplet-based assays, in accordance with aspects of the present disclosure.

FIG. 2 is a schematic view of an exemplary system for performing the assay of FIG. 1.

FIG. 3 is an isometric view of an illustrative assay performance system suitable for performing assays including aqueous sample stabilization in accordance with aspects of the present disclosure.

FIG. 4 is a flow chart showing steps of an illustrative method for performing assays, including sample stabilization in accordance with aspects of the present disclosure.

FIG. 5 is a graph of amplification data collected on samples stored at room temperature for 20 hours in droplets (Panel A) or in bulk (Panel B).

FIG. 6 is a graph of amplification data collected on sample converted into droplets and then thermocycled immediately (Panel A) or stored for 20 hours at room temperature and then thermocycled (Panel B).

### DETAILED DESCRIPTION

Various aspects and examples of an assay performance system including aqueous sample stabilization, as well as related methods, are described below and illustrated in the associated drawings. Some or all of the assay performance system may be automated, as described below. Unless otherwise specified, an assay performance system in accordance with aspects of the present disclosure, and/or its various components may, but are not required to, contain at least one of the structure, components, functionality, and/or variations described, illustrated, and/or incorporated herein. Furthermore, the process steps, structures, components, functionalities, and/or variations described, illustrated, and/or incorporated herein in connection with the present teachings may, but are not required to, be included in other similar assay performance systems and methods. The following description of various examples is merely illustrative in nature and is in no way intended to limit the disclosure, its application, or uses. Additionally, the advantages provided by the examples and embodiments described below are illustrative in nature and not all examples and embodiments provide the same advantages or the same degree of advantages.

This Detailed Description includes the following sections, which follow immediately below: (1) Definitions; (2) Overview; (3) Examples, Components, and Alternatives; (4) Illustrative Combinations and Additional Examples; (5) Advantages, Features, and Benefits; and (6) Conclusion. The Examples, Components, and Alternatives section is further divided into subsections A through F, each of which is labeled accordingly.

### Definitions

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional, unrecited elements or method steps.

Terms such as “first”, “second”, and “third” are used to distinguish or identify various members of a group, or the like, and are not intended to show serial or numerical limitation.

“Coupled” means connected, either permanently or releasably, whether directly or indirectly through intervening components.

“AKA” means “also known as,” and may be used to indicate an alternative or corresponding term for a given element or elements.

Technical terms used in this disclosure have the meanings that are commonly recognized by those skilled in the art. However, the following terms may have additional meanings, as described below.

Emulsion: a composition comprising liquid droplets disposed in an immiscible carrier fluid, which also is liquid. The carrier fluid, also termed a background fluid, forms a continuous phase, which may be termed a carrier phase, a carrier, and/or a background phase. The droplets (e.g., aqueous droplets) are formed by at least one droplet fluid, also termed a foreground fluid, which is a liquid and which forms a droplet phase (which may be termed a dispersed phase or discontinuous phase). The droplet phase is immiscible with the continuous phase, which means that the droplet phase (i.e., the droplets) and the continuous phase (i.e., the carrier fluid) do not mix to attain homogeneity. The droplets are isolated from one another by the continuous phase and encapsulated (i.e., enclosed/surrounded) by the continuous phase.

The droplets of an emulsion may have any uniform or non-uniform distribution in the continuous phase. If non-uniform, the concentration of the droplets may vary to provide one or more regions of higher droplet density and one or more regions of lower droplet density in the continuous phase. For example, droplets may sink or float in the continuous phase, may be clustered in one or more packets along a channel, may be focused toward the center or perimeter of a flow stream, or the like. When droplets are said to be “suspended in the background fluid,” this is intended to cover all of these possibilities.

Any of the emulsions disclosed herein may be monodisperse, that is, composed of droplets of at least generally uniform size, or may be polydisperse, that is, composed of droplets of various sizes. If monodisperse, the droplets of the emulsion may, for example, vary in volume by a standard deviation that is less than about plus or minus 100%, 50%, 20%, 10%, 5%, 2%, or 1% of the average droplet volume. Droplets generated from an orifice may be monodisperse or polydisperse.

An emulsion may have any suitable composition. The emulsion may be characterized by the predominant liquid compound or type of liquid compound in each phase. The predominant liquid compounds in the emulsion may be water and oil. “Oil” is any liquid compound or mixture of liquid compounds that is immiscible with water and that has a high content of carbon. In some examples, oil also may have a high content of hydrogen, fluorine, silicon, oxygen, or any combination thereof, among others. For example, any of the emulsions disclosed herein may be a water-in-oil (W/O) emulsion (i.e., aqueous droplets in a continuous oil phase). The oil may, for example, be or include at least one silicone oil, mineral oil, fluorocarbon oil, vegetable oil, or a combination thereof, among others. Any other suitable components may be present in any of the emulsion phases, such as at least one surfactant, reagent, sample (i.e., partitions thereof), other additive, label, particles, or any combination thereof.

Emulsions may become unstable when heated (e.g., to temperatures above 60° C.) when they are in a packed state (e.g., each droplet is near a neighboring droplet), because heat generally lowers interfacial tensions, which can lead to droplet coalescence. Thus, packed emulsions may not maintain their integrity during high-temperature reactions, such as PCR, unless emulsion droplets are kept out of contact with one another or additives (e.g., other oil bases, surfactants, etc.) are used to modify the stability conditions (e.g., interfacial tension, viscosity, steric hindrance, etc.). For example, the droplets may be arranged in single file and spaced from one another along a channel to permit thermal cycling in order to perform PCR. However, following this approach using a typical emulsion does not permit a high density of droplets, thereby substantially limiting throughput in droplet-based assays.

Any emulsion disclosed herein may be a heat-stable emulsion. A heat-stable emulsion is any emulsion that resists coalescence when heated to at least 50° C. A heat-stable emulsion may be a PCR-stable emulsion, which is an emulsion that resists coalescence throughout the thermal cycling of PCR (e.g., to permit performance of digital PCR). Accordingly, a PCR-stable emulsion may be resistant to coalescence when heated to at least 80° C. or 90° C., among others. Due to heat stability, a PCR-stable emulsion, in contrast to a standard emulsion, enables PCR assays to be performed in droplets that remain substantially monodisperse throughout thermal cycling. Accordingly, digital PCR assays with PCR-stable emulsions may be substantially more quantitative than with standard emulsions. An emulsion may be formulated as PCR stable by, for example, proper selection of carrier fluid and surfactants, among others. An exemplary oil formulation to generate PCR-stable emulsions for flow-through assays is as follows: (1) Dow Corning 5225C Formulation Aid (10% active ingredient in decamethylcyclopentasiloxane)—20% w/w, 2% w/w final concentration active ingredient, (2) Dow Corning 749 Fluid (50% active ingredient in decamethylcyclopentasiloxane)—5% w/w, 2.5% w/w active ingredient, and (3) Poly(dimethylsiloxane) Dow Corning 200® fluid, viscosity 5.0 cSt (25° C.)—75% w/w. An exemplary oil formulation to generate PCR-stable emulsions for batch assays is as follows: (1) Dow Corning 5225C Formulation Aid (10% active ingredient in decamethylcyclopentasiloxane)—20% w/w, 2% w/w final concentration active ingredient, (2) Dow Corning 749 Fluid (50% active ingredient in decamethylcyclopentasiloxane)—60% w/w, 30% w/w active ingredient, and (3) Poly(dimethylsiloxane) Dow Corning 200® fluid, viscosity 5.0 cSt (25° C.)—20% w/w. Other suitable formulations may be used. For example, suitable formulations based on fluorinated oil chemistry are disclosed in U.S. patent application Ser. No. 12/976,827, the entirety of which is incorporated herein for all purposes.

Partition: a separated portion of a bulk volume. The partition may be a sample partition generated from a sample, such as a prepared sample, that forms the bulk volume. Partitions generated from a bulk volume may be substantially uniform in size or may have distinct sizes (e.g., sets of partitions of two or more discrete, uniform sizes). Exemplary partitions are droplets. Partitions may also vary continuously in size with a predetermined size distribution or with a random size distribution.

Droplet: a small volume of liquid, typically with a spherical shape, encapsulated by an immiscible fluid, such as a continuous phase of an emulsion. The volume of a droplet, and/or the average volume of droplets in an emulsion, may, for example, be less than about one microliter (i.e., a “microdroplet”) (or between about one microliter and one nanoliter or between about one microliter and one picoliter), less than about one nanoliter (or between about one nanoliter and one picoliter), or less than about one picoliter (or between about one picoliter and one femtoliter), among others. A droplet (or droplets of an emulsion) may have a diameter (or an average diameter) of less than about 1000, 100, or 10 micrometers, or of about 1000 to 10 micrometers, among others. A droplet may be spherical or nonspherical. A droplet may be a simple droplet or a compound droplet, that is, a droplet in which at least one droplet encapsulates at least one other droplet.

Surfactant: a surface-active agent capable of reducing the surface tension of a liquid in which it is dissolved, and/or the interfacial tension with another phase. A surfactant, which also or alternatively may be described as a detergent and/or a wetting agent, incorporates both a hydrophilic portion and a hydrophobic portion, which collectively confer a dual hydrophilic-lipophilic character on the surfactant. A surfactant may be characterized according to a Hydrophile-Lipophile Balance (HLB) value, which is a measure of the surfactant's hydrophilicity compared to its lipophilicity. HLB values range from 0-60 and define the relative affinity of a surfactant for water and oil. Nonionic surfactants generally have HLB values ranging from 0-20 and ionic surfactants may have HLB values of up to 60. Hydrophilic surfactants have HLB values greater than about 10 and a greater affinity for water than oil. Lipophilic surfactants have HLB values less than about 10 and a greater affinity for oil than water. The emulsions disclosed herein and/or any phase thereof, may include at least one hydrophilic surfactant, at least one lipophilic surfactant, or a combination thereof. Alternatively, or in addition, the emulsions disclosed herein and/or any phase thereof, may include at least one nonionic (and/or ionic) detergent. Furthermore, an emulsion disclosed herein and/or any phase thereof may include a surfactant comprising polyethyleneglycol, polypropyleneglycol, or Tween 20, among others.

Packet: a set of droplets or other isolated partitions disposed in the same continuous volume or volume region of a continuous phase. A packet thus may, for example, constitute all of the droplets of an emulsion or may constitute a segregated fraction of such droplets at a position along a channel. Typically, a packet refers to a collection of droplets that when analyzed in partial or total give a statistically relevant sampling to quantitatively make a prediction regarding a property of the entire starting sample from which the initial packet of droplets was made. The packet of droplets also indicates a spatial proximity between the first and the last droplets of the packet in a channel.

As an analogy with information technology, each droplet serves as a “bit” of information that may contain sequence specific information from a target analyte within a starting sample. A packet of droplets is then the sum of all these “bits” of information that together provide statistically relevant information on the analyte of interest from the starting sample. As with a binary computer, a packet of droplets is analogous to the contiguous sequence of bits that comprises the smallest unit of binary data on which meaningful computations can be applied. A packet of droplets can be encoded temporally and/or spatially relative to other packets that are also disposed in a continuous phase (such as in a flow stream), and/or with the addition of other encoded information (optical, magnetic, etc.) that uniquely identifies the packet relative to other packets.

Test: a procedure(s) and/or reaction(s) used to characterize a sample, and any signal(s), value(s), data, and/or result(s) obtained from the procedure(s) and/or reaction(s). A test also may be described as an assay. Exemplary droplet-based assays are biochemical assays using aqueous assay mixtures. More particularly, the droplet-based assays may be enzyme assays and/or binding assays, among others. The enzyme assays may, for example, determine whether individual droplets contain a copy of a substrate molecule (e.g., a nucleic acid target) for an enzyme and/or a copy of an enzyme molecule. Based on these assay results, a concentration and/or copy number of the substrate and/or the enzyme in a sample may be estimated.

Reaction: a chemical reaction, a binding interaction, a phenotypic change, or a combination thereof, which generally provides a detectable signal (e.g., a fluorescence signal) indicating occurrence and/or an extent of occurrence of the reaction. An exemplary reaction is an enzyme reaction that involves an enzyme-catalyzed conversion of a substrate to a product.

Any suitable enzyme reactions may be performed in the droplet-based assays disclosed herein. For example, the reactions may be catalyzed by a kinase, nuclease, nucleotide cyclase, nucleotide ligase, nucleotide phosphodiesterase, polymerase (DNA or RNA), prenyl transferase, pyrophospatase, reporter enzyme (e.g., alkaline phosphatase, beta-galactosidase, chloramphenicol acetyl transferse, glucuronidase, horse radish peroxidase, luciferase, etc.), reverse transcriptase, topoisomerase, etc.

Sample: a compound, composition, and/or mixture of interest, from any suitable source(s). A sample is the general subject of interest for a test that analyzes an aspect of the sample, such as an aspect related to at least one analyte that may be present in the sample. Samples may be analyzed in their natural state, as collected, and/or in an altered state, for example, following storage, preservation, extraction, lysis, dilution, concentration, purification, filtration, mixing with one or more reagents, pre-amplification (e.g., to achieve target enrichment by performing limited cycles (e.g., <15) of PCR on sample prior to PCR), removal of amplicon (e.g., treatment with uracil-d-glycosylase (UDG) prior to PCR to eliminate any carry-over contamination by a previously generated amplicon (i.e., the amplicon is digestable with UDG because it is generated with dUTP instead of dTTP)), partitioning, or any combination thereof, among others. Clinical samples may include nasopharyngeal wash, blood, plasma, cell-free plasma, buffy coat, saliva, urine, stool, sputum, mucous, wound swab, tissue biopsy, milk, a fluid aspirate, a swab (e.g., a nasopharyngeal swab), and/or tissue, among others. Environmental samples may include water, soil, aerosol, and/or air, among others. Research samples may include cultured cells, primary cells, bacteria, spores, viruses, small organisms, any of the clinical samples listed above, or the like. Additional samples may include foodstuffs, weapons components, biodefense samples to be tested for bio-threat agents, suspected contaminants, and so on.

Samples may be collected for diagnostic purposes (e.g., the quantitative measurement of a clinical analyte such as an infectious agent) or for monitoring purposes (e.g., to determine that an environmental analyte of interest such as a bio-threat agent has exceeded a predetermined threshold). A sample that is in liquid form or that has been mixed into a liquid may be referred to as a sample fluid.

Analyte: a component(s) or potential component(s) of a sample that is analyzed in a test. An analyte is a specific subject of interest in a test where the sample is the general subject of interest. An analyte may, for example, be a nucleic acid, protein, peptide, enzyme, cell, bacteria, spore, virus, organelle, macromolecular assembly, drug candidate, lipid, carbohydrate, metabolite, or any combination thereof, among others. An analyte may be tested for its presence, activity, and/or other characteristic in a sample and/or in partitions thereof. The presence of an analyte may relate to an absolute or relative number, concentration, binary assessment (e.g., present or absent), or the like, of the analyte in a sample or in one or more partitions thereof. In some examples, a sample may be partitioned such that a copy of the analyte is not present in all of the partitions, such as being present in the partitions at an average concentration of about 0.0001 to 10,000, 0.001 to 1000, 0.01 to 100, 0.1 to 10, or one copy per partition.

Reagent: a compound, set of compounds, and/or composition that is combined with a sample in order to perform a particular test(s) on the sample. A reagent may be a target-specific reagent, which is any reagent composition that confers specificity for detection of a particular target(s) or analyte(s) in a test. A reagent optionally may include a chemical reactant and/or a binding partner for the test. A reagent may, for example, include at least one nucleic acid, protein (e.g., an enzyme), cell, virus, organelle, macromolecular assembly, potential drug, lipid, carbohydrate, inorganic substance, or any combination thereof, and may be an aqueous composition, among others. In exemplary embodiments, the reagent may be an amplification reagent, which may include at least one primer or at least one pair of primers for amplification of a nucleic acid target, at least one probe and/or dye to enable detection of amplification, a polymerase, nucleotides (dNTPs and/or NTPs), divalent magnesium ions, potassium chloride, buffer, or any combination thereof, among others.

Nucleic acid: a compound comprising a chain of nucleotide monomers. A nucleic acid may be single-stranded or double-stranded (i.e., base-paired with another nucleic acid), among others. The chain of a nucleic acid may be composed of any suitable number of monomers, such as at least about ten or one-hundred, among others. Generally, the length of a nucleic acid chain corresponds to its source, with synthetic nucleic acids (e.g., primers and probes) typically being shorter, and biologically/enzymatically generated nucleic acids (e.g., nucleic acid analytes) typically being longer.

A nucleic acid may have a natural or artificial structure, or a combination thereof. Nucleic acids with a natural structure, namely, deoxyribonucleic acid (DNA) and ribonucleic acid (RNA), generally have a backbone of alternating pentose sugar groups and phosphate groups. Each pentose group is linked to a nucleobase (e.g., a purine (such as adenine (A) or guanine (T)) or a pyrimidine (such as cytosine (C), thymine (T), or uracil (U))). Nucleic acids with an artificial structure are analogs of natural nucleic acids and may, for example, be created by changes to the pentose and/or phosphate groups of the natural backbone. Exemplary artificial nucleic acids include glycol nucleic acids (GNA), peptide nucleic acids (PNA), locked nucleic acid (LNA), threose nucleic acids (TNA), and the like.

The sequence of a nucleic acid is defined by the order in which nucleobases are arranged along the backbone. This sequence generally determines the ability of the nucleic acid to bind specifically to a partner chain (or to form an intramolecular duplex) by hydrogen bonding. In particular, adenine pairs with thymine (or uracil) and guanine pairs with cytosine. A nucleic acid that can bind to another nucleic acid in an antiparallel fashion by forming a consecutive string of such base pairs with the other nucleic acid is termed “complementary.”

Replication: a process forming a copy (i.e., a direct copy and/or a complementary copy) of a nucleic acid or a segment thereof. Replication generally involves an enzyme, such as a polymerase and/or a ligase, among others. The nucleic acid and/or segment replicated is a template (and/or a target) for replication.

Amplification: a reaction in which replication occurs repeatedly over time to form multiple copies of at least one segment of a template molecule. Amplification may generate an exponential or linear increase in the number of copies as amplification proceeds. Typical amplifications produce a greater than 1,000-fold increase in copy number and/or signal. Exemplary amplification reactions for the droplet-based assays disclosed herein may include the polymerase chain reaction (PCR) or ligase chain reaction, each of which is driven by thermal cycling. The droplet-based assays also or alternatively may use other amplification reactions, which may be performed isothermally, such as branched-probe DNA assays, cascade-RCA, helicase-dependent amplification, loop-mediated isothermal amplification (LAMP), nucleic acid based amplification (NASBA), nicking enzyme amplification reaction (NEAR), PAN-AC, Q-beta replicase amplification, rolling circle replication (RCA), self-sustaining sequence replication, strand-displacement amplification, and the like. Amplification may utilize a linear or circular template.

Amplification may be performed with any suitable reagents. Amplification may be performed, or tested for its occurrence, in an amplification mixture, which is any composition capable of generating multiple copies of a nucleic acid target molecule, if present, in the composition. An amplification mixture may include any combination of at least one primer or primer pair, at least one probe, at least one replication enzyme (e.g., at least one polymerase, such as at least one DNA and/or RNA polymerase), and deoxynucleotide (and/or nucleotide) triphosphates (dNTPs and/or NTPs), among others. Further aspects of assay mixtures and detection strategies that enable multiplexed amplification and detection of two or more target species in the same droplet are described elsewhere herein.

PCR: nucleic acid amplification that relies on alternating cycles of heating and cooling (i.e., thermal cycling) to achieve successive rounds of replication. PCR may be performed by thermal cycling between two or more temperature set points, such as a higher melting (denaturation) temperature and a lower annealing/extension temperature, or among three or more temperature set points, such as a higher melting temperature, a lower annealing temperature, and an intermediate extension temperature, among others. PCR may be performed with a thermostable polymerase, such as Taq DNA polymerase (e.g., wild-type enzyme, a Stoffel fragment, FastStart polymerase, etc.), Pfu DNA polymerase, S-Tbr polymerase, Tth polymerase, Vent polymerase, or a combination thereof, among others. PCR generally produces an exponential increase in the amount of a product amplicon over successive cycles.

Any suitable PCR methodology or combination of methodologies may be utilized in the droplet-based assays disclosed herein, such as allele-specific PCR, assembly PCR, asymmetric PCR, digital PCR, endpoint PCR, hot-start PCR, in situ PCR, intersequence-specific PCR, inverse PCR, linear after exponential PCR, ligation-mediated PCR, methylation-specific PCR, miniprimer PCR, multiplex ligation-dependent probe amplification, multiplex PCR, nested PCR, overlap-extension PCR, polymerase cycling assembly, qualitative PCR, quantitative PCR, real-time PCR, RT-PCR, single-cell PCR, solid-phase PCR, thermal asymmetric interlaced PCR, touchdown PCR, or universal fast walking PCR, among others.

Digital PCR: PCR performed on portions of a sample to determine the linkage of DNA segments, or the presence/absence, concentration, and/or copy number of a nucleic acid target in the sample, based on how many of the sample portions support amplification of the target. Digital PCR may (or may not) be performed as endpoint PCR. Digital PCR may (or may not) be performed as real-time PCR for each of the partitions.

PCR theoretically results in an exponential amplification of a nucleic acid sequence (analyte) from a sample. By measuring the number of amplification cycles required to achieve a threshold level of amplification (as in real-time PCR), one can theoretically calculate the starting concentration of nucleic acid. In practice, however, there are many factors that make the PCR process non-exponential, such as varying amplification efficiencies, low copy numbers of starting nucleic acid, and competition with background contaminant nucleic acid. Digital PCR is generally insensitive to these factors, since it does not rely on the assumption that the PCR process is exponential. In digital PCR, individual nucleic acid molecules are separated from the initial sample into partitions, then amplified to detectable levels. Each partition then provides digital information on the presence or absence of each individual nucleic acid molecule within each partition. When enough partitions are measured using this technique, the digital information can be consolidated to make a statistically relevant measure of starting concentration for the nucleic acid target (analyte) in the sample.

The concept of digital PCR may be extended to other types of analytes, besides nucleic acids. In particular, a signal amplification reaction may be utilized to permit detection of a single copy of a molecule of the analyte in individual droplets, to permit data analysis of droplet signals for other analytes (e.g., using an algorithm based on Poisson statistics). Exemplary signal amplification reactions that permit detection of single copies of other types of analytes in droplets include enzyme reactions.

Qualitative PCR: a PCR-based analysis that determines whether or not a target is present in a sample, generally without any substantial quantification of target presence. In exemplary embodiments, digital PCR that is qualitative may be performed by determining whether a packet of droplets contains at least a predefined percentage of positive droplets (a positive sample) or not (a negative sample).

Quantitative PCR: a PCR-based analysis that determines a degree of linkage, or a concentration and/or copy number of a target in a sample.

RT-PCR (reverse transcription-PCR): PCR utilizing a complementary DNA template produced by reverse transcription of RNA. RT-PCR permits analysis of an RNA sample by (1) forming complementary DNA copies of RNA, such as with a reverse transcriptase enzyme, and (2) PCR amplification using the complementary DNA as a template. In some embodiments, the same enzyme, such as Tth polymerase, may be used for reverse transcription and PCR.

Real-time PCR: a PCR-based analysis in which amplicon formation is measured during the reaction, such as after completion of one or more thermal cycles prior to the final thermal cycle of the reaction. Real-time PCR generally provides quantification of a target based on the kinetics of target amplification.

Endpoint PCR: a PCR-based analysis in which amplicon formation is measured after the completion of thermal cycling.

Amplicon: a product of an amplification reaction. An amplicon may be single-stranded or double-stranded, or a combination thereof. An amplicon corresponds to any suitable segment or the entire length of a nucleic acid target.

Primer: a nucleic acid capable of, and/or used for, priming replication of a nucleic acid template. Thus, a primer is a shorter nucleic acid that is complementary to a longer template. During replication, the primer is extended, based on the template sequence, to produce a longer nucleic acid that is a complementary copy of the template. A primer may be DNA, RNA, an analog thereof (i.e., an artificial nucleic acid), or any combination thereof. A primer may have any suitable length, such as at least about 10, 15, 20, or 30 nucleotides. Exemplary primers are synthesized chemically. Primers may be supplied as at least one pair of primers for amplification of at least one nucleic acid target. A pair of primers may be a sense primer and an antisense primer that collectively define the opposing ends (and thus the length) of a resulting amplicon.

Probe: a nucleic acid connected to at least one label, such as at least one dye. A probe may be a sequence-specific binding partner for a nucleic acid target and/or amplicon. The probe may be designed to enable detection of target amplification based on fluorescence resonance energy transfer (FRET). An exemplary probe for the nucleic acid assays disclosed herein includes one or more nucleic acids connected to a pair of dyes that collectively exhibit fluorescence resonance energy transfer (FRET) when proximate one another. The pair of dyes may provide first and second emitters, or an emitter and a quencher, among others. Fluorescence emission from the pair of dyes changes when the dyes are separated from one another, such as by cleavage of the probe during primer extension (e.g., a 5′ nuclease assay, such as with a TAQMAN probe), or when the probe hybridizes to an amplicon (e.g., a molecular beacon probe).

The nucleic acid portion of the probe may have any suitable structure or origin, for example, the portion may be a locked nucleic acid, a member of a universal probe library, or the like. In other cases, a probe and one of the primers of a primer pair may be combined in the same molecule (e.g., AMPLIFLUOR primers or SCORPION primers). As an example, the primer-probe molecule may include a primer sequence at its 3′ end and a molecular beacon-style probe at its 5′ end. With this arrangement, related primer-probe molecules labeled with different dyes can be used in a multiplexed assay with the same reverse primer to quantify target sequences differing by a single nucleotide (single nucleotide polymorphisms (SNPs)). Another exemplary probe for droplet-based nucleic acid assays is a Plexor primer.

Label: an identifying and/or distinguishing marker or identifier connected to or incorporated into any entity, such as a compound, biological particle (e.g., a cell, bacteria, spore, virus, or organelle), or droplet. A label may, for example, be a dye that renders an entity optically detectable and/or optically distinguishable. Exemplary dyes used for labeling are fluorescent dyes (fluorophores) and fluorescence quenchers.

Reporter: a compound or set of compounds that reports a condition, such as the extent of a reaction. Exemplary reporters comprise at least one dye, such as a fluorescent dye or an energy transfer pair, and/or at least one oligonucleotide. Exemplary reporters for nucleic acid amplification assays may include a probe and/or an intercalating dye (e.g., SYBR Green, ethidium bromide, etc.).

Code: a mechanism for differentiating distinct members of a set. Exemplary codes to differentiate different types of droplets may include different droplet sizes, dyes, combinations of dyes, amounts of one or more dyes, enclosed code particles, or any combination thereof, among others. A code may, for example, be used to distinguish different packets of droplets, or different types of droplets within a packet, among others.

Binding partner: a member of a pair of members that bind to one another. Each member may be a compound or biological particle (e.g., a cell, bacteria, spore, virus, organelle, or the like), among others. Binding partners may bind specifically to one another. Specific binding may be characterized by a dissociation constant of less than about 10 4, 10 6, 10 8, or 10 10 M. Exemplary specific binding partners include biotin and avidin/streptavidin, a sense nucleic acid and a complementary antisense nucleic acid (e.g., a probe and an amplicon), a primer and its target, an antibody and a corresponding antigen, a receptor and its ligand, and the like.

### Overview

In general, an assay performance system in accordance with the present teachings may include one or more automated steps and/or assay performance assemblies. The inventor has found that at least some of the bulk aqueous samples involved in such systems may be stabilized by emulsification, as opposed to storage in bulk form, even at relatively high temperatures. This result goes against the expectation that, over time, a component or multiple components of the aqueous phase of the emulsion could be recruited to an emulsification's droplet surfaces. If this were to happen, the amount of surface area provided by the droplet interfaces is relatively large for the volume of aqueous phase present. Depending on the component(s) recruited to the droplet surfaces, substantial inhibition of reactions (such as PCR) could potentially occur. Accordingly, systems and aspects of the methods disclosed herein may incorporate emulsification at an early stage of the assay process, even for samples that would normally remain stored or queued in bulk form before processing.

The present disclosure provides systems, including apparatus and methods, for performing assays. These systems may involve, among others, (A) preparing one or more samples, such as clinical or environmental samples, for analysis, (B) separating components of the samples by partitioning them into droplets or other partitions, each containing only about one component (such as a single copy of a nucleic acid target

(DNA or RNA) or other analyte of interest), (C) amplifying or otherwise reacting the components within the droplets, (D) detecting the amplified or reacted components, or characteristics thereof, and/or (E) analyzing the resulting data. In this way, complex samples may be converted into a plurality of simpler, more easily analyzed samples, with concomitant reductions in background and assay times.

### Examples, Components, and Alternatives

The following sections describe selected aspects of exemplary assay performance systems, as well as related systems and/or methods. The examples in these sections are intended for illustration and should not be interpreted as limiting the entire scope of the present disclosure. Each section may include one or more distinct embodiments or examples, and/or contextual or related information, function, and/or structure.

### A. Illustrative Assay Method

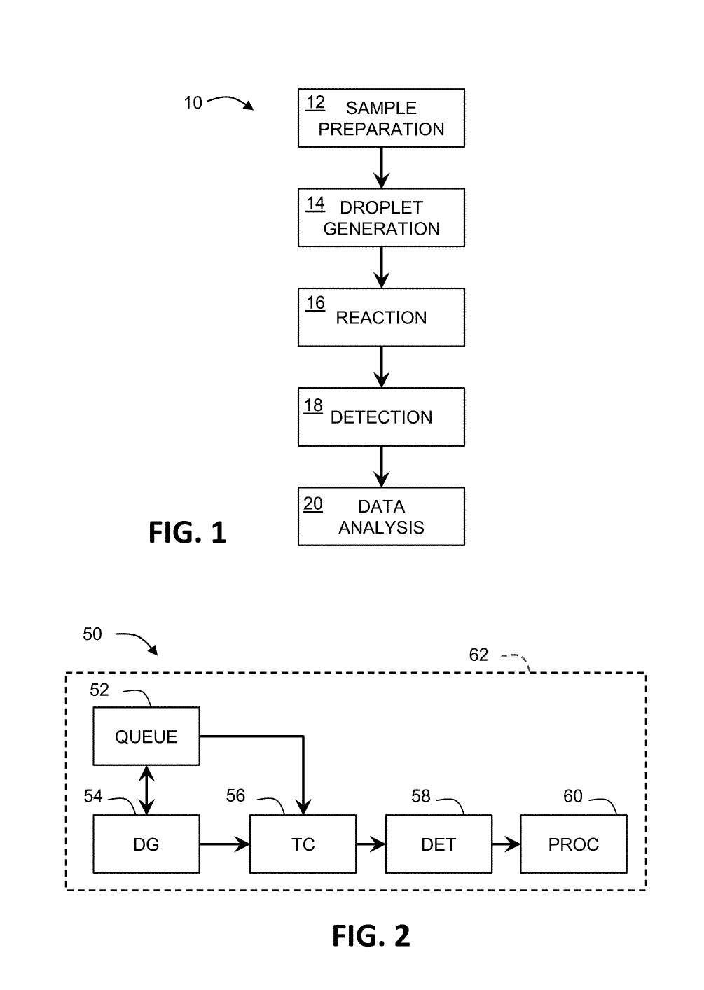

As shown in FIG. 1, this section describes an illustrative assay method 10. FIG. 1 is a flowchart illustrating steps performed in an illustrative method, and may not recite the complete process or all steps of the method. Although various steps of method 10 are described below and depicted in FIG. 1 the steps need not necessarily all be performed, and in some cases may be performed simultaneously or in a different order than the order shown.

FIG. 1 shows an exemplary method/system 10 for performing a droplet- or partition-based assay. In brief, method 10 may include sample preparation 12, partitioning or droplet generation 14, reaction (e.g., amplification) 16, detection 18, and data analysis 20. The system may be utilized to perform a digital PCR (polymerase chain reaction) analysis. The steps presented for method 10 may be performed in any suitable order and in any suitable combination. Furthermore, the steps may be combined with and/or modified by any other suitable steps, aspects, and/features of the present disclosure or of the materials in the Cross References.

More specifically, sample preparation 12 may involve collecting a sample, such as a clinical or environmental sample, treating the sample to release associated nucleic acids, and forming a reaction mixture involving the nucleic acids (e.g., for amplification of a target nucleic acid). Preparation of the sample may include any suitable manipulation of the sample, such as collection, dilution, concentration, purification, lyophilization, freezing, extraction, combination with one or more assay reagents, performance of at least one preliminary reaction to prepare the sample for one or more reactions in the assay, or any combination of these, among others. Preparation of the sample may include rendering the sample competent for subsequent performance of one or more reactions, such as one or more enzyme catalyzed reactions and/or binding reactions.

In some embodiments, preparation of the sample may include combining the sample with reagents for amplification and for reporting whether or not amplification occurred. Such reagents may include any combination of primers for the targets (e.g., a forward primer and a reverse primer for each target), reporters (e.g., probes) for detecting amplification of the targets, dNTPs and/or NTPs, at least one enzyme (e.g., a polymerase, a ligase, a reverse transcriptase, or a combination thereof, each of which may or may not be heat-stable), or the like. Accordingly, preparation of the sample may render the sample (or partitions thereof) capable of amplification of each of one or more targets, if present, in the sample (or a partition thereof).

Partitioning 14 may involve separating or distributing any suitable portion (or all) of the sample to the partitions. Each partition may include a fluid volume that is isolated from the fluid volumes of other partitions. The partitions may be isolated from one another by a carrier fluid, such as a continuous phase of an emulsion, by a solid phase, such as at least one wall of a container, or a combination thereof, among others. In some embodiments, the partitions may be droplets disposed in a continuous phase, such that the droplets and the continuous phase collectively form an emulsion. This form of partitioning may be referred to as droplet generation. Droplet generation may involve encapsulating the nucleic acids in droplets, for example, with about one copy of each target nucleic acid per droplet, where the droplets are suspended in an immiscible carrier fluid, such as oil, to form the emulsion.

The partitions may be formed by any suitable procedure, in any suitable manner, and with any suitable properties. For example, the partitions may be formed with a fluid dispenser, such as a pipette, with a droplet generator, by agitation of the sample (e.g., shaking, stirring, sonication, etc.), or the like. Accordingly, the partitions may be formed serially, in parallel, or in batch. The partitions may have any suitable volume or volumes. The partitions may be of substantially uniform volume or may have different volumes. Exemplary partitions having substantially the same volume are monodisperse droplets. Exemplary volumes for the partitions include an average volume of less than about 100, 10 or 1 μL, less than about 100, 10, or 1 nL, or less than about 100, 10, or 1 pL, among others.

The partitions, when formed, may be competent for performance of one or more reactions in the partitions. Alternatively, one or more reagents may be added to the partitions after they are formed to render them competent for reaction. The reagents may be added by any suitable mechanism, such as a fluid dispenser, fusion of droplets, or the like. Any of the reagents may be combined with the partitions (or a bulk phase sample) in a macrofluidic or microfluidic environment.

It may be desirable, in systems such as DNA amplification systems, among others, to generate sample-containing droplets using a partially or completely disposable apparatus. This may be accomplished by a disposable cartridge configured to generate droplets as part of a series of sample preparation steps that also may include lysing, purification, and concentration, among others. However, in other cases, it may be desirable to provide a partially or completely disposable apparatus configured to perform droplet generation without performing substantial additional sample preparation steps. This may be desirable, for example, when the DNA amplification system is configured to analyze samples that are typically prepared at another location or by a practitioner. Under these circumstances, a dedicated droplet generation system may be the simplest and most economical solution.

The components of droplet generation systems described herein may include, for example, substrates, wells (i.e. reservoirs), channels, tubes, and the like, which may be assembled, for example, in the form of a unitary cartridge. These components may be manufactured by any suitable method(s) known in the art, for example by injection molding, machining, hot embossing, and/or the like. In some cases, all of the components of a droplet generation system disclosed according to the present teachings may be proprietary. In other cases, one or more components of a disclosed system may be available as an off-the-shelf component, which may be integrated with other components either with or without modification.

Many configurations of droplet generators may be suitable as components of a droplet generation system according to the present teachings. For example, suitable droplet generators include butted tubes, tubes drilled or otherwise formed with intersecting channels, tubes partially or completely inserted inside other tubes, and tubes having multiple apertures, among others, where “tubes” means elongate hollow structures of any cross-sectional shape. Suitable fluid reservoirs include pipette tips, spin columns, wells (either individual or in a plate array), tubes, and syringes, among others.

Reaction 16 may involve subjecting the droplets to a suitable reaction or reactions, such as thermal cycling (also referred to as thermocycling) to induce PCR amplification, so that target nucleic acids, if any, within the droplets are amplified to form additional copies. Each reaction performed may occur selectively (and/or substantially) in only a subset of the partitions, such as less than about one-half, one-fourth, or one-tenth of the partitions, among others. The reaction may involve a target, which may, for example, be a template and/or a reactant (e.g., a substrate), and/or a binding partner, in the reaction. The reaction may occur selectively (or selectively may not occur) in partitions containing at least one copy of the target.

The reaction may or may not be an enzyme-catalyzed reaction. In some examples, the reaction may be an amplification reaction, such as PCR and/or ligase chain reaction. Accordingly, a plurality of amplification reactions for a plurality of targets may be performed simultaneously in the partitions.

Performing a reaction may include subjecting the partitions to one or more conditions that promote occurrence of the reaction. The conditions may include heating the partitions and/or incubating the partitions at a temperature above room temperature. In some examples, the conditions may include thermally cycling the partitions to promote a polymerase chain reaction and/or ligase chain reaction.

Detection 18 may involve detecting some signal(s) from the droplets indicative of whether or not there was amplification. Finally, data analysis 20 may involve estimating a concentration of the target nucleic acid in the sample based on the percentage of droplets in which amplification occurred.

### B. Illustrative Schematic Assay System

As shown in the schematic diagram of FIG. 2, this section describes an illustrative system 50 for performing the assays of FIG. 1. System 50 includes a queuing portion 52 for storing and/or handling samples, a partitioning assembly in the form of a droplet generator 54 (“DG”), a thermal incubation assembly, in the form of a thermocycler 56 (“TC”), a detection assembly (a detector) 58 (“DET”), and a data processing assembly 60 (“PROC”) (also referred to as a processor), among possible others. One or more, or all, of these components and instruments may be housed or otherwise assembled into one or more groupings or assemblies. For example, all of the components and instruments may form a single assay performance assembly 62, as indicated in FIG. 2. In other examples, system 50 may be separated into pre-thermocycling, thermocycling, and post-thermocycling sub-assemblies.

Data processor 60 may be, or may be included in, a controller that communicates with and controls operation of any suitable combination of the assemblies or sub-assemblies. The arrows between the assemblies indicate optional, and in some cases automatic, movement or transfer, such as movement or transfer of fluid (e.g., a continuous phase of an emulsion) and/or partitions (e.g., droplets) or signals/data. For example, a cartridge or plate having wells or reservoirs of fluids and/or emulsions may be automatically or manually transferred between instruments. In some examples, one or more of the operations described with respect to FIG. 1 may be performed on such a cartridge by the same (e.g., multi-function) instrument or assembly. Any suitable combination of the components may be operatively connected to one another, and/or one or more of the assemblies may be unconnected to the other assemblies, such that, for example, material/data is transferred manually.

System 50 generally operates as follows. One or more cartridges or other sample-containing receptacles are loaded into queuing portion 52. From queuing portion 52, a cartridge (or multiple cartridges) are transferred to droplet generator 54, e.g., automatically, such as by a pick-and-place, conveyance, carousel, or other suitable apparatus. Droplet generator 54 forms droplets disposed in a carrier fluid, such as a continuous phase, substantially as described above with respect to FIG. 1. The cartridge may then be transported to the thermocycler or, in some cases, back to the queuing portion. The droplets are cycled thermally with thermocycler 56 to promote amplification of targets in the droplets. Composite signals are detected from the droplets with detector 58. The signals are processed by processor 60 to calculate levels of the targets.

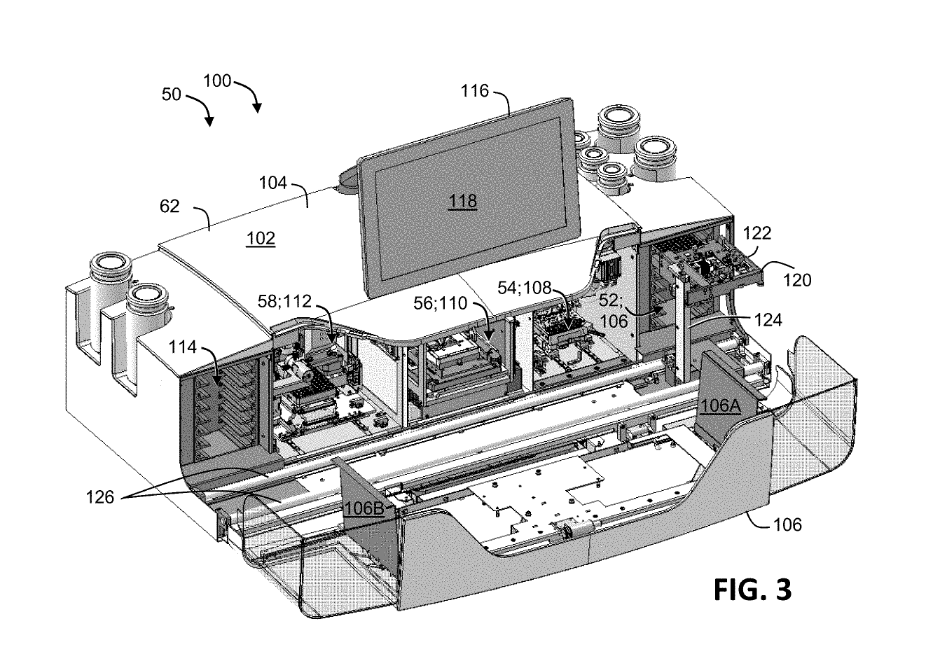

In some examples, multiple cartridges or plates containing aqueous samples and/or carrier fluid(s) may be loaded into queuing portion 52 at once. For example, devices and instruments subsequent to queuing portion 52 may be partially or fully automatic. Accordingly, a plurality of plates may be loaded into system 50 by a user or operator, then cycled through the component instruments or operations of system 50 without further intervention. In some examples, thermocycling using thermocycler 56 may generally be the bottleneck, constraint, or rate-limiting step in system 50. Thermocycling can take significantly longer than other operations, e.g., approximately two hours. For that reason, plates loaded into system 50 may be required to wait several hours (e.g., twenty hours or more when loading ten plates) before thermocycling. Droplet generation enhances reaction stability as compared with bulk storage. Therefore, cartridges may be essentially immediately cycled through droplet generator 54 upon loading the system. Related methods are described in further detail below, with respect to FIG. 3.

Further aspects of sample preparation, droplet generation, assays, reagents, reactions, thermal cycling, detection, and data processing, among others, that may be suitable for the methods and systems disclosed herein, are described below and in the documents listed above under Cross-References, which are incorporated herein by reference. Additional aspects are disclosed in PCT Patent Application Publication No. WO 2011/120006 A1, published Sep. 29, 2011; and PCT Patent Application Publication No. WO 2011/120024 A1, the entireties of which are also hereby incorporated by reference.

### C. Illustrative Assay System

As shown in FIG. 3, this section describes an illustrative system 100 for performing the assays of FIG. 1. System 100 is an example of system 50, described above.

System 100 includes a single assay performance assembly, with several subassemblies or stations contained in a common housing 102. Housing 102 includes a main housing 104 and a pivoting door section 106 configured to be opened for accessing the internal subassemblies of system 100, as shown in FIG. 3. Pivoting door 106 may be configured to provide simultaneous access to two or more of the subassemblies of system 100. In this example, pivoting door 106 provides selected simultaneous access to all subassemblies, and also includes internal walls 106A and 106B to keep the input and output queues separated from other subassemblies.

Within housing 102, and as viewed from right to left in FIG. 3, are the following subassemblies: a queuing or hotel portion 106 (corresponding to queuing portion 52), a droplet generator portion 108 (corresponding to DG 54), a thermocycler portion 110 (corresponding to TC 56), a detection portion 112 (corresponding to detector 58), and an output queue portion 114. A display 116 is mounted to system 100 for providing a graphical user interface (GUI) 118 configured to permit a user to interact with system 100 and its processor (not shown).

In this example, sample-containing receptacles (AKA cartridges) are loaded into hotel 106 and retained on shelves therein. An automatic handler 120 (also referred to as a pick-and-place handler) transports the cartridges between the subassemblies of system 100. Handler 120 includes an extendable gripper portion 122 configured to move horizontally into and out of each station, and to ride up and down on a vertical mast 124 (e.g., driven by a motorized lead screw). Mast 124 is configured to be moved horizontally (e.g., by a belt drive) on a pair of horizontal rails 126. Accordingly, handler 120 can move the cartridges in all three dimensions, into and out of each of the stations of system 100.

From hotel 106, a cartridge (or multiple cartridges) are automatically transferred to droplet generator 108 by handler 120. Droplet generator 108 forms droplets disposed in a carrier fluid, such as a continuous phase, substantially as described above with respect to FIG. 1. The cartridge is automatically transferred by handler 120 to thermocycler 110, where the droplets are cycled thermally (e.g., multiple times) to promote amplification of targets in the droplets. The cartridge is then automatically transferred by handler 120 to detector portion 112, where composite signals are detected from the droplets. The signals are processed by the processor of system 100 to calculate levels of the targets. Following detection and processing, each cartridge is then transferred to output queue portion 114 for additional processing or disposal. In some examples, as described below, handler 120 may transport multiple cartridges from hotel 106 to droplet generator 108 and back into the hotel, e.g., while the first cartridge is thermocycling. These cartridges would then be moved directly from the hotel to the DG portion when their turn comes, then on to the other stations as described above.

In some examples, wall 106A and/or 106B may be selectively retractable, to permit the handler to travel freely when needed. In other words, either or both of the internal walls may be transitionable between an extended configuration, in which the wall separates the input queue from the droplet generation module, and a retracted configuration, in which the wall is pivoted to permit free movement of the automatic sample plate transport device (in this example, the handler). In some examples, the handler or other transport device is configured to transition the internal wall between the extended configuration and the retracted configuration. For example, the internal wall may be biased toward the extended configuration (e.g., by a hinge-mounted torsion spring) and the handler overcomes that bias by pushing the wall outboard when needed to access the input queue or output queue.

### D. Illustrative Assay Performance Method Including Sample Stabilization

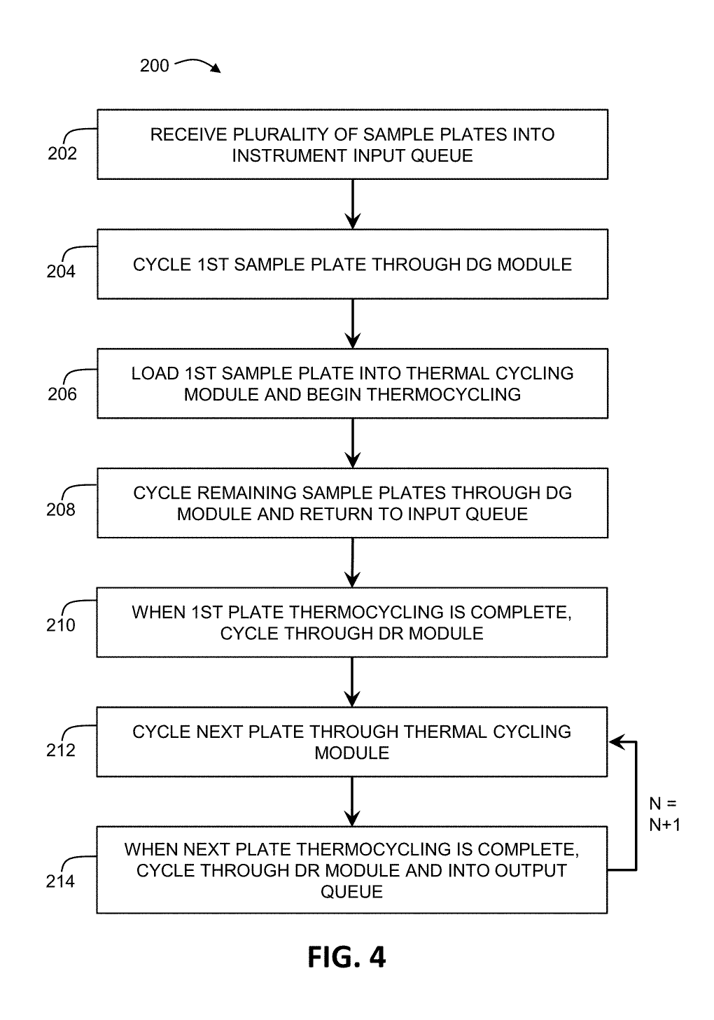

This section describes steps of an illustrative method for performing assays, including stabilization of a sample; see FIG. 4. Aspects of assay performance systems described above, such as systems 50 and/or 100, may be utilized in the method steps described below. Where appropriate, reference may be made to previously described components and systems that may be used in carrying out each step. These references are for illustration, and are not intended to limit the possible ways of carrying out any particular step of the method.

FIG. 4 is a flowchart illustrating steps performed in an illustrative method, and may not recite the complete process or all steps of the method. Although various steps of method 200 are described below and depicted in FIG. 4, the steps need not necessarily all be performed, and in some cases may be performed simultaneously or in a different order than the order shown.

At step 202, an automated assay performance system (e.g., system 50 or 100) may receive a plurality of sample plates into an instrument input queue. For example, a plurality (e.g., seven) sample-containing cartridges or plates may be loaded into queuing portion 52 of system 50. This queuing portion may include any suitable storage compartment or arrangement, such as a rack, carousel, container, and/or the like, or any combination of these. Hotel 106 is a suitable example of the queuing portion.

At step 204, a first one of the sample plates may be cycled through a droplet generation (DG) module or instrument (e.g., DG 54 or 108). For example, a first of the cartridges loaded into system 50 may be automatically transferred to DG assembly 54, where emulsification is performed. For example, the cartridge may be transferred automatically by handler 120.

At step 206, the first sample plate is loaded into a thermal cycling module (e.g., TC 56 or 110) and thermocycling is commenced. For example, the cartridge containing emulsifications/droplets may be automatically loaded into thermocycling assembly 56 or 110 (e.g., by handler 120). As described above, thermocycling may take over an hour to complete (e.g., approximately two hours).

At step 208, while awaiting completion of thermocycling of the first plate, remaining sample plates may be cycled through the droplet generation module and returned to the input queue or hotel. For example, if seven cartridges were loaded in step 202, step 208 may include performing droplet generation on cartridges two through seven in DG assembly 54 while cartridge one was in the TC process, then returning each cartridge to queuing portion 52. In some examples, this step includes sequentially placing each cartridge (AKA sample plate) into the DG module and performing droplet generation. In some examples, droplet generation may be performed on multiple cartridges or plates in parallel.

At step 210, when thermocycling of the first emulsified samples is complete, the first plate is transferred to a droplet reader (DR) module (e.g., DR 58 or 112). For example, the first cartridge may be automatically transferred from TC assembly 56 or 110 to detection assembly 58 or 112 (e.g., by handler 120). This operation makes room in the thermocycler for a next plate or cartridge.

At step 212, a next plate is cycled through the thermal cycling module. For example, the second cartridge may be transferred automatically from queuing portion 52 to TC assembly 56 for cycling. This second cartridge may have been in storage for approximately two hours. Note that wait times for following cartridges will rise in multiples of the thermocycling cycle time. For example, the third cartridge may wait four hours before thermocycling, the fourth may wait six hours, and so on, until the final of seven cartridges would be waiting at least approximately twelve hours. This highlights the importance of early droplet generation/emulsification, such that the aqueous samples may be stabilized for the lengthy wait times.

At step 214, when the thermocycling of step 212 is complete, the plate in the TC module may be cycled through the droplet reader (e.g., module 58 or 112) and into an output queue (or simply to an output) (e.g., output queue 114). For example, the second cartridge may finish thermocycling, then be automatically transferred to the detection assembly 58 of system 50, and then exit the system. Essentially, because the constraint of the system is the TC module, each subsequent/downstream operation can be carried out as soon as the next cartridge or plate is finished thermocycling. Accordingly, subsequent plates will finish steps 212 and 214 in sequence, as indicated on FIG. 4.

Although stabilization has been incorporated or “built in” to the systems described by method 200, stabilization of aqueous samples may be performed in other systems or for other purposes.

### E. Illustrative Data

This section describes exemplary experiments conducted to demonstrate the efficacy and performance benefits of the systems and methods described herein. In brief, these experiments show that samples stored in droplets before thermocycling are more stable than samples stored in bulk. This improved stability enhances the quality of data collected from the samples, which, in turn, increases the accuracy of assays performed on the samples and conclusions drawn from those assays. The experiments were performed using a variety of samples (e.g., S. aureus, GC-rich assays, and AT-rich assays), fluorophores (e.g., EvaGreen, FAM, and HEX), temperatures (e.g., 4° C., room temperature, and 37° C.), and storage times (e.g., none, overnight, and 20 hours).

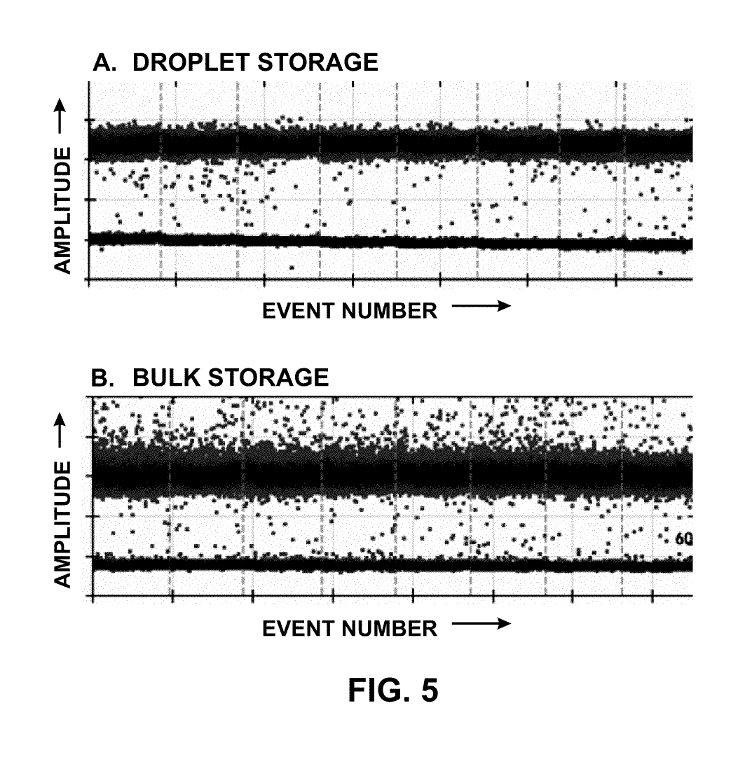

FIG. 5 is a graph of amplification data collected on samples stored at room temperature for 20 hours in droplets (Panel A) or in bulk (Panel B). The droplet samples were then thermocycled and analyzed. The bulk samples were then converted to droplets, thermocycled, and analyzed. Both sets of samples included Staphylococcus aureus (S. aureus) and EvaGreen. Droplets positive for sample have higher amplitudes; droplets negative for sample have lower (e.g., zero) amplitudes. In these experiments, data from the sample stored as droplets was more tightly clustered and less noisy than data from sample stored in bulk. Thus, when sample must be stored, it is better stored after droplet formation.

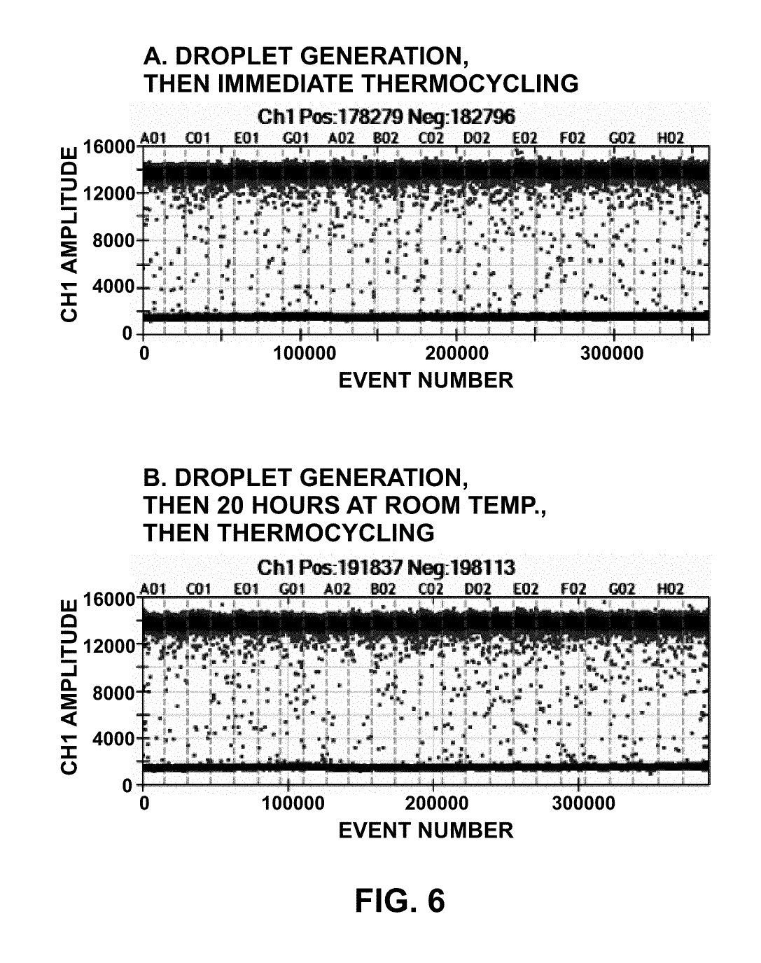

FIG. 6 is a graph of amplification data collected on sample converted into droplets and then thermocycled immediately (Panel A) or stored for 20 hours at room temperature and then thermocycled (Panel B). Qualitatively, the data look essentially identical. Quantitatively, the data are essentially identical: the fraction of positive droplets to all droplets is 49.4% for immediate thermocycling and 49.2% for thermocycling after 20 hours of storage. Thus, sample stored in droplets is quite stable. This result applied for different samples, different fluorophores, and different storage conditions (e.g., cooled, room temperature, and warmed).

Additional data is presented in U.S. Provisional Patent Application Ser. No. 62/451,004, filed Jan. 26, 2017, which is incorporated herein by reference.

### F. Illustrative Combinations and Additional Examples

This section describes additional aspects and features of assay performance systems and methods of the present disclosure, presented without limitation as a series of paragraphs, some or all of which may be alphanumerically designated for clarity and efficiency. Each of these paragraphs can be combined with one or more other paragraphs, and/or with disclosure from elsewhere in this application, including the materials incorporated by reference in the Cross-References, in any suitable manner. Some of the paragraphs below expressly refer to and further limit other paragraphs, providing without limitation examples of some of the suitable combinations.

A0. A method for conducting assays, the method comprising (1) receiving a plurality of aqueous sample-containing sample plates into an input queue; (2) stabilizing an aqueous sample on a first one of the sample plates by automatically cycling the first one of the sample plates through a droplet generation module; (3) automatically loading the first one of the sample plates into a thermal cycling module and beginning thermocycling of the first one of the sample plates; (4) while the first one of the sample plates is thermocycling, stabilizing aqueous samples on all remaining sample plates by automatically cycling the remaining sample plates through the droplet generation module and automatically returning each of the remaining sample plates to the input queue; (5) when thermocycling of the first one of the sample plates is complete, automatically cycling the first one of the sample plates through a droplet reader module; and (6) in response to completion of thermocycling of the first one of the sample plates, sequentially automatically cycling each of the remaining sample plates through the thermal cycling module and the droplet reader module.

A1. The method of A0, wherein the plurality of sample plates includes less than ten sample plates.

A2. The method of A1, wherein the input queue has space for up to seven sample plates.

A3. The method of A0, wherein the thermal cycling module takes approximately two hours to thermocycle each sample plate.

A4. The method of A0, further comprising automatically transporting the first one of the sample plates from the input queue to the droplet generator.

A5. The method of A4, wherein automatically transporting the first one of the sample plates is performed using an automated pick-and-place sample plate handler.

A6. The method of A0, wherein the input queue, the droplet generation module, the thermal cycling module, and the droplet reader module are all contained in a common housing.

A7. The method of A6, wherein the common housing has a single pivotable door configured to provide simultaneous user access to the input queue, the droplet generation module, the thermal cycling module, and the droplet reader module.

A8. The method of A6, wherein the pivotable door includes an internal wall transitionable between an extended configuration, in which the wall separates the input queue from the droplet generation module, and a retracted configuration, in which the wall is pivoted to permit free movement of an automatic sample plate transport device.

A9. The method of A8, wherein the automatic sample plate transport device is configured to transition the internal wall between the extended configuration and the retracted configuration.

A10. The method of A8, wherein the internal wall is biased toward the extended configuration.

B0. A method for conducting assays, the method comprising (1) receiving a plurality of aqueous sample-containing sample plates into an input queue of an assay performance assembly having an automated sample plate transport device; (2) stabilizing an aqueous sample on a first one of the sample plates by automatically transporting the first one of the sample plates to a droplet generation module of the assay performance assembly using the sample plate transport device and cycling the first one of the sample plates through the droplet generation module to generate an emulsion in the first one of the sample plates; (3) automatically loading the first one of the sample plates into a thermal cycling module of the assay performance assembly using the sample plate transport device and beginning thermocycling of the first one of the sample plates; (4) while the first one of the sample plates is thermocycling, using the sample plate transport device to stabilize aqueous samples on all remaining sample plates by automatically cycling the remaining sample plates through the droplet generation module and automatically returning each of the remaining sample plates to the input queue; (5) when thermocycling of the first one of the sample plates is complete, automatically cycling the first one of the sample plates through a droplet reader module of the assay performance assembly using the sample plate transport device; and (6) in response to completion of thermocycling of the first one of the sample plates, sequentially automatically cycling each of the remaining sample plates through the thermal cycling module and the droplet reader module using the sample plate transport device.

B1. The method of B0, wherein automatically returning each of the remaining sample plates to the input queue includes repositioning a retractable internal wall of the assay performance assembly to permit access to the input queue.

B2. The method of B1, wherein the retractable internal wall is repositioned by way of interaction with the sample plate transport device.

B3. The method of B0, wherein the input queue has space for up to seven sample plates.

B4. The method of B0, wherein the thermal cycling module takes approximately two hours to thermocycle each sample plate.

B5. The method of B0, wherein the input queue, the droplet generation module, the thermal cycling module, and the droplet reader module are all contained in a common housing.

B6. The method of B5, wherein the common housing has a single pivotable door configured to provide simultaneous user access to the input queue, the droplet generation module, the thermal cycling module, and the droplet reader module.

C0. A process for performing assays, the process comprising (1) preparing an aqueous sample; (2) stabilizing the aqueous sample by generating an emulsification including partitions of the aqueous sample surrounded by a carrier fluid; (3) waiting at least two hours while allowing the emulsification to reach a temperature of no more than approximately 33 degrees Celsius; and (4) facilitating a polymerase chain reaction by thermally cycling the emulsification.

C1. The process of C0, wherein the waiting step includes waiting more than approximately ten hours.

C2. The process of C0, wherein the carrier fluid comprises an oil.

C3. The process of C0, wherein the emulsification is performed using a droplet generator.

D0. A process for stabilizing bulk aqueous samples, the process including (1) partitioning an aqueous sample by emulsifying the sample using a carrier fluid; and (2) storing the emulsified sample for more than approximately two hours.

D1. The process of D0, wherein the emulsified sample is stored for more than approximately ten hours.

E0. A method for conducting assays, the method comprising (1) receiving a plurality of aqueous sample-containing sample plates into an instrument input queue; (2) stabilizing an aqueous sample on a first one of the sample plates by cycling the first one of the sample plates through a droplet generation module; (3) loading the first one of the sample plates into a thermal cycling module and beginning thermocycling of the first one of the sample plates; (4) while the first one of the sample plates is thermocycling, stabilizing aqueous samples on all remaining sample plates by cycling all the remaining sample plates through the droplet generation module and returning each of the remaining sample plates to the input queue; (5) when thermocycling of the first one of the sample plates is complete, cycling the first one of the sample plates through a droplet reader module; and (6) in response to completion of thermocycling of the first one of the sample plates, sequentially cycling each of the remaining sample plates through the thermal cycling module and the droplet reader module.

E1. The method of E0, wherein the plurality of sample plates includes approximately ten sample plates.

E2. The method of E0, wherein the instrument input queue has space for up to ten sample plates.

E3. The method of E0, wherein the thermal cycling module takes approximately two hours to thermocycle each sample plate.

F0. A system for performing assays, the system including (1) an input queuing portion for receiving a plurality of aqueous sample cartridges; (2) a droplet generator for emulsifying aqueous samples contained in the sample cartridges; (3) a thermocycler for thermally cycling the emulsified samples to promote a polymerase chain reaction (PCR); (4) a detection apparatus for detecting markers indicating that the PCR step was successful; and (5) a cartridge handling system coupled to the queuing portion and configured to automatically transfer cartridges from the input queuing portion to the droplet generator and from the droplet generator to the input queuing portion.

F1. The system of F0, further including a controller in communication with the droplet generator, the thermocycler, the detection apparatus, and the cartridge handling system, such that the controller causes the transfer of sample cartridges between systems.

F2. The system of F1, wherein the controller is configured to cycle each of the remaining aqueous sample cartridges through the droplet generator while a first cartridge cycles through the thermocycler.

F3. The system of F0, further comprising at least seven aqueous sample cartridges capable of being stored simultaneously in the queuing portion.

G0. Reaction may be set up as follows:

G1. Thaw all components to room temperature. Mix vigorously by vortexing the tubes at the maximum speed for 30 sec to ensure homogeneity, because a concentration gradient may form during −20 C storage. Centrifuge briefly to collect contents at the bottom of the tube.

G2. Prepare samples at the desired concentration before setting up the reaction mix.

H0. Sample cartridges may be set up as follows:

H1. Pipette and load 20 ul reaction mix with samples into sample holes of the cartridges.

H2 Seal the cartridges with a plate sealer (e.g., Bio-Rad's PX1 PCR Plate Sealer, Catalog# 1814000) and a foil seal.

H2A. Set up the sealing condition of the PX1 PCR Plate Sealer at 180 C for 0.5 seconds

H2B. Touch the button to open the drawer

H2C. Place a block in the drawer

H2D. Place the cartridge in the block

H2E. Place the seal on the cartridge

H2F. Touch the button to close the drawer

H2G. Touch “Seal” button when the set temperature has been reached and the seal button is green. When sealing is complete, the drawer will automatically open

H2H. Remove the cartridge, leaving the block. Rotate the cartridge 180° and place the cartridge in the block again

H2I. Touch the button to close the drawer and touch the seal button

H2J. Remove the cartridge from the block. Remove the block from the drawer

H2K. Centrifuge the cartridge for 30 seconds at 1,000 rpm

H3. Load the sealed cartridge into the PCR system (e.g., QX ONE ddPCR System).

### Advantages, Features, and Benefits

The different embodiments and examples of the assay performance systems and methods described herein provide several advantages over known solutions. For example, illustrative embodiments and examples described herein use emulsification (i.e., droplet generation) to enhance reaction stability of stored samples, as compared with bulk storage.

Additionally, and among other benefits, illustrative embodiments and examples described herein allow enhanced stabilization of reaction up to temperatures of approximately 33 C, if not higher. Accordingly, bulk samples may be emulsified prior to general storage.

Additionally, and among other benefits, illustrative embodiments and examples described herein allow another benefit of droplet formation that has to do with the geometries of DG cartridges and the like. For example, a DG cartridge may include a so-called sipper or other tube configured to draw up samples and/or carrier fluid during the DG process. Because these sipper tubes have a small diameter, capillary action may cause fluids to be drawn into the sipper(s) without applying an external source of vacuum or pressure. Accordingly, and especially over time, undesired and/or unmanaged or unexpected mixing of components may occur due to the capillary action. For this reason, it is again advantageous to emulsify the samples before storage.

No known system or device can perform these functions. However, not all embodiments and examples described herein provide the same advantages or the same degree of advantage.

### Conclusion

The disclosure set forth above may encompass multiple distinct examples with independent utility. Although each of these has been disclosed in its preferred form(s), the specific embodiments thereof as disclosed and illustrated herein are not to be considered in a limiting sense, because numerous variations are possible. To the extent that section headings are used within this disclosure, such headings are for organizational purposes only. The subject matter of the disclosure includes all novel and nonobvious combinations and subcombinations of the various elements, features, functions, and/or properties disclosed herein. The following claims particularly point out certain combinations and subcombinations regarded as novel and nonobvious. Other combinations and subcombinations of features, functions, elements, and/or properties may be claimed in applications claiming priority from this or a related application. Such claims, whether broader, narrower, equal, or different in scope to the original claims, also are regarded as included within the subject matter of the present disclosure.

What is claimed is:

1. A method for conducting assays, the method comprising:
receiving a plurality of aqueous sample-containing sample plates into an input queue;
stabilizing an aqueous sample on a first one of the sample plates by automatically cycling the first one of the sample plates through a droplet generation module;
automatically loading the first one of the sample plates into a thermal cycling module and beginning thermocycling of the first one of the sample plates;
while the first one of the sample plates is thermocycling, stabilizing aqueous samples on all remaining sample plates by automatically cycling the remaining sample plates through the droplet generation module and automatically returning each of the remaining sample plates to the input queue;
when thermocycling of the first one of the sample plates is complete, automatically cycling the first one of the sample plates through a droplet reader module; and
in response to completion of thermocycling of the first one of the sample plates, sequentially automatically cycling each of the remaining sample plates through the thermal cycling module and the droplet reader module.

2. The method of claim 1, wherein the input queue has space for up to seven sample plates.

3. The method of claim 1, further comprising automatically transporting the first one of the sample plates from the input queue to the droplet generator.

4. The method of claim 3, wherein automatically transporting the first one of the sample plates is performed using an automated pick-and-place sample plate handler.

5. The method of claim 1, wherein the input queue, the droplet generation module, the thermal cycling module, and the droplet reader module are all contained in a common housing.

6. The method of claim 5, wherein the common housing has a single pivotable door configured to provide simultaneous user access to the input queue, the droplet generation module, the thermal cycling module, and the droplet reader module.

7. The method of claim 6, wherein the pivotable door includes an internal wall transitionable between an extended configuration, in which the wall separates the input queue from the droplet generation module, and a retracted configuration, in which the wall is pivoted to permit free movement of an automatic sample plate transport device.

8. The method of claim 7, wherein the automatic sample plate transport device is configured to transition the internal wall between the extended configuration and the retracted configuration.

9. The method of claim 7, wherein the internal wall is biased toward the extended configuration.

10. A method for conducting assays, the method comprising:
receiving a plurality of aqueous sample-containing sample plates into an input queue of an assay performance assembly having an automated sample plate transport device;
stabilizing an aqueous sample on a first one of the sample plates by automatically transporting the first one of the sample plates to a droplet generation module of the assay performance assembly using the sample plate transport device and cycling the first one of the sample plates through the droplet generation module to generate an emulsion in the first one of the sample plates;
automatically loading the first one of the sample plates into a thermal cycling module of the assay performance assembly using the sample plate transport device and beginning thermocycling of the first one of the sample plates;
while the first one of the sample plates is thermocycling, using the sample plate transport device to stabilize aqueous samples on all remaining sample plates by automatically cycling the remaining sample plates through the droplet generation module and automatically returning each of the remaining sample plates to the input queue;
when thermocycling of the first one of the sample plates is complete, automatically cycling the first one of the sample plates through a droplet reader module of the assay performance assembly using the sample plate transport device; and
in response to completion of thermocycling of the first one of the sample plates, sequentially automatically cycling each of the remaining sample plates through the thermal cycling module and the droplet reader module using the sample plate transport device.

11. The method of claim 10, wherein automatically returning each of the remaining sample plates to the input queue includes repositioning a retractable internal wall of the assay performance assembly to permit access to the input queue.

12. The method of claim 11, wherein the retractable internal wall is repositioned by way of interaction with the sample plate transport device.

13. The method of claim 10, wherein the input queue has space for up to seven sample plates.

14. The method of claim 10, wherein the input queue, the droplet generation module, the thermal cycling module, and the droplet reader module are all contained in a common housing.

15. The method of claim 14, wherein the common housing has a single pivotable door configured to provide simultaneous user access to the input queue, the droplet generation module, the thermal cycling module, and the droplet reader module.

16. A system for performing assays, the system including:
an input queuing portion for receiving a plurality of aqueous sample cartridges;
a droplet generator for emulsifying aqueous samples contained in the sample cartridges;
a thermocycler for thermally cycling the emulsified samples to promote a polymerase chain reaction (PCR);
a detection apparatus for detecting markers indicating that the PCR step was successful; and
a cartridge handling system coupled to the queuing portion and configured to automatically transfer cartridges from the input queuing portion to the droplet generator and from the droplet generator to the input queuing portion.

17. The system of claim 16, further including a controller in communication with the droplet generator, the thermocycler, the detection apparatus, and the cartridge handling system, such that the controller causes the transfer of sample cartridges between systems.

18. The system of claim 17, wherein the controller is configured to cycle each of the remaining aqueous sample cartridges through the droplet generator while a first cartridge cycles through the thermocycler.

19. The system of claim 16, further comprising at least seven aqueous sample cartridges capable of being stored simultaneously in the queuing portion.
